(19) United States
(12) Patent Application Publication
(10) Pub. No.: US 2019/0094244 A1
(43) Pub. Date: Mar. 28, 2019

(54) Free Functional Annexin Levels in Plasma as a Biomarker of Cardiovascular Risk

(71) Applicants: INSERM (Institut National de la Sante et de la Recherche Medicale), Paris (FR); Universite Paris Descartes, Paris (FR)

(72) Inventor: Olivier P. BLANC-BRUDE, Paris (FR)

(21) Appl. No.: 16/087,183

(22) PCT Filed: Mar. 22, 2017
(86) PCT No.: PCT/EP2017/056784

§ 371 Date: Sep. 21, 2018

(30) Foreign Application Priority Data
Mar. 22, 2016 (EP) 16305320.0

(51) Int. Cl. G01N 33/68; A61K 38/17

(52) U.S. Cl. CPC G01N 33/6893; A61K 38/1709; G01N 2800/50; G01N 2405/04; G01N 2800/32; G01N 2333/4718

(57) ABSTRACT

The present invention relates to a method for diagnosing the occurrence of a vascular dysfunction or a vascular injury in a subject, said method comprising a step consisting of determining in a plasma sample obtained from said subject the level of free annexin. The present invention further relates to a method for determining whether a subject is at risk for severe vasculopathy, cardiovascular complications and cardiovascular disease, said method comprising a step consisting of determining in a plasma sample obtained from said subject the level of free annexin. Preferably, the methods further comprise the steps consisting of determining the level of circulating phosphatidylserine positive (PS+) microparticles (MPs) in the plasma sample obtained from said subject and calculating the ratio free annexin/circulating PS+MPs. The invention also relates to a kit for use in a method according to the invention and to a phosphatidylserine antagonist, for use in a method of treatment of severe vasculopathy, cardiovascular complications and cardiovascular disease in a subject having a decreased level of free annexin as compared to a free annexin reference level, wherein the method comprises a determination of the free annexin level in a plasma sample of said subject. Preferably, a phosphatidylserine antagonist is for use in a method of treatment of severe vasculopathy, cardiovascular complications and cardiovascular disease in a subject having a decreased ratio of free annexin/circulating PS+MPs as compared to a free annexin/circulating PS−MPs reference ratio, wherein the method further comprises the steps consisting of determining the circulating PS+MPs level in said plasma sample and calculating the free annexin/circulating P8−MPs ratio.

### FIELD OF THE INVENTION

The present invention relates to a method for diagnosing and stratifying a subject at risk for cardiovascular disease.

### BACKGROUND OF THE INVENTION

According to the World Health Organization (WHO; Geneva) cardiovascular diseases (CVDs) are the number one cause of death globally: more people die annually from CVDs than from any other cause. An estimated 17.5 million people died from CVDs in 2012, representing 31% of all global deaths. Of these deaths, an estimated 7.4 million were due to coronary heart disease and 6.7 million were due to stroke. Over three quarters of CVD deaths take place in low- and middle-income countries. Out of the 16 million deaths under the age of 70 due to non-communicable diseases, 82% are in low and middle income countries and 37% are caused by CVDs. In addition, many cardiovascular incidents are not necessarily fatal, but may impair the ability to live a normal daily life, resulting in enormous healthcare costs to society.

Most cardiovascular diseases can be prevented by addressing behavioural risk factors such as tobacco use, unhealthy diet and obesity, physical inactivity and by using population-wide strategies. People with cardiovascular disease, or who are at high cardiovascular risk (due to the presence of one or more risk factors such as hypertension, diabetes, hyperlipidaemia or already established disease) need early detection and management using counselling and medicines, as appropriate.

The high incidence and mortality rates of cardiovascular diseases have stimulated extensive investments by both the biotechnology and pharmaceutical industries.

However, an efficient treatment and prevention of cardiovascular diseases does not only involve the administration of appropriate medicaments but requires also reliable diagnostic tools. Therefore the identification and use of molecular markers of cardiovascular diseases for early patient diagnosis, stratification and risk prevention in a patient is of major clinical importance.

Extracellular vesicles (EVs) are subcellular membrane vesicles that are released by multiple cell types in the body, retain a phospholipid membrane and are particularly accessible to quantification and characterization in the blood stream. Microparticles (MPs) are a subgroup of EVs ranging in size from 0.1 to 1.0 μm. They can be released by platelets, red blood cells (RBCs) and vascular cells, or by multiple other activated or apoptotic tissues. The majority of EVs are thought to expose externalized anionic phospholipid phosphatidylserine (PS) at their surface, as well as surface membrane antigens representative of their cellular origin. It is now well recognized that EVs, and MPs in particular, behave as vectors of bioactive molecules, playing a role in blood coagulation, inflammation, cell activation, immunomodulation, cancer growth and metastasis. In clinical practice, circulating EVs originating from blood and vascular cells are elevated in a variety of prothrombotic and inflammatory disorders, cardiovascular diseases, autoimmune conditions, infectious diseases and cancer (Lacroix R et al., 2013 J Thromb Haemost. April 2. doi: 10.1111).

The detection of EVs in blood and circulating plasma is particularly relevant to cardiovascular disease pathogenesis because failure to clear vesicles notably leads to an increase in the circulating levels of proatherogenic factors. In addition, ineffective EV release could lead to localized cell damage (Augustine D et al., 2014, Circ Res. 114(1):109-13).

Indeed the presence of circulating MPs in plasma has been recently associated with adverse clinical events in the cardiovascular field such as, for example, higher risks for transfusion accidents, myocardial infarction, coronary atherothrombosis, atherosclerotic plaques, acute coronary syndromes, hypertension or hypertriglyceridemia (Augustine D et al., Circ Res. 2014, 114(1):109-13; Jy Wet al., J Thorac Surg. 2015, 149(1):305-11; Giannopoulos G et al., Int J Cardiol. 2014, 176(1):145-50; Morel et al., Atherosclerosis 2009, 204(2):636-41; Empana J P et al., Eur Heart J Acute Cardiovasc. Care 2015, 4(1):28-36; Ferreira A C et al. Circulation. 2004, 110(23):3599-603; Wang J. M. et al. J. Hum. Hypertens. 2009, 23:307-315).

Amongst patients at higher risk of cardiovascular disease, are patients suffering from chronic hemolytic anemiae, including sickle cell disease (SCD). Intravascular hemolysis and the breakdown of erythrocytes in SCD induce an increased release of “free” hemoglobin, heme and erythrocyte MPs expressing phosphatidylserine into the bloodstream. This increased release of MPs has been shown to exacerbate vascular injury, which may further trigger chronic degenerative manifestations through ischemic events and functional vascular remodelling. Indeed, in vitro MPs trigger the production of radical oxygen species by endothelial monolayers, favour erythrocyte adhesion, and induce endothelial apoptosis. MPs also compromise vasodilatation in perfused microvessels (Camus S M et al., Blood 2012, 120(25):5050-58; Wun T et al., J Lab Clin Med. 1997, 129(5):507-16). Circulating MP levels have also been shown to further increase during vaso-occlusive crises (VOCs) versus steady state, as revealed by the detection of phosphatidylserine positive (PS+) MPs (Camus S M et al., Blood 2012, 120(25):5050-58). VOCs participate in recurrent ischemic tissue injury on a chronic basis resulting in progressive organ damage, multiple organ failure and cardiovascular damages.

The significant variability in the degree of change in MP levels between individuals after cardiovascular stress has raised the interesting possibility that MPs could be a predictive biomarker (Augustine D et al., Circ Res. 2014, 114(1):109-13; Berezin A E et al., Int J Clin Exp Med. 2015, 8(10): 18255-18264; Burger D et al., Clinical Science 2013, 124, 423-41; Camus S M et al., Blood 2015, 125(24):3805-14, but see also WO 2012/120130).

Therefore there remains a need for more specific biomarkers which would efficiently allow the diagnosis of subjects at risk for cardiovascular disease, and their stratification, and for predicting a patient's outcome in cardiovascular diseases with optimal decremented potency. More generally there remains a need for identifying a biomarker usable in stratified medicine allowing to stage individuals according to the risk that they are exposed to, for further vascular injury and cardiovascular diseases.

### SUMMARY OF THE INVENTION

In healthy subjects, circulating microparticles expressing phosphatidylserine (PS+MPs) are targeted by annexins and annexin-A5 in particular. Annexins are intracellular proteins that associate with membrane during cell stress, and which can also be found in plasma. Annexins act usually as specific phosphatidylserine (PS) inhibitors, neutralizing PS-mediated effects of PS externalized by stressed cells and MPs. Annexins, and annexin-A5 in particular, are generally thought to be anti-inflammatory and anti-thrombotic protective agents. Annexin levels in circulating plasma have been demonstrated to increase in stress conditions and notably after the onset of cardiovascular events. However, little is known about the manner in which plasma annexins circulate, their molecular partners and the mechanism of their presentation to target membranes.

Annexins and annexin-A5 in particular, can be measured in patient plasma, using commercial kits based on enzyme-linked immunosorbent assay (ELISA) technology. This approach allows the measurement of total annexin levels, whichever their molecular partners may be.

The inventors have now demonstrated that there also exists a fraction of annexins, and of annexin-A5 in particular, that remains free, functional and capable of engaging new PS+ membranes or MPs. This free annexin level, and free annexin-A5 level in particular, can be readily detected in plasma samples from control healthy subjects using the technology described herein.

The inventors have also further demonstrated that equilibrium between PS externalization in membranes and circulating MPs, and the plasma levels of annexins can be determined, leaving a fraction of free annexins not engaged with membranes. However, this equilibrium can be compromised in patients with vascular dysfunctions, such as SCD, obese and/or diabetic patients for example, which are at high vascular risk. The increase in PS externalization in membranes and circulating MPs on one hand, and the fixed or decreasing levels of plasma annexins on the other hand can trigger a complete consumption of plasma annexins by PS+ membranes and MPs, and therefore an apparent depletion in free annexins. The inventor's results show that free circulating plasma annexins appear to be entirely consumed by excess PS externalization in patients at high cardiovascular risk. In those conditions, plasma annexins are therefore insufficient to neutralize the high levels of PS+MPs and PS+ membranes, which are produced, for instance, by stressed RBCs during hemolysis.

The inventors also showed that the levels of circulating PS+MPs were positively correlated with the severity of vascular dysfunction. Indeed, PS+MPs were significantly increased in SCD patients, compared to control healthy subjects, and they rose again significantly in SCD patients at the early phase of vaso-occlusive crises (VOCs).

These results illustrate that the assessment of MP (or EV) levels alone, or of total annexin levels in plasma, according to current practice, reflect quite imperfectly the real cardiovascular risk and the clinical outcome of the patient for the purpose of diagnosis or prognosis of cardiovascular or inflammatory conditions. Indeed, the presence of circulating MPs could be fully offset by increased annexin levels, since the MPs would become invisible by annexin labelling and FACS. On the contrary, high levels of circulating plasma annexins (total) may hide the fact that annexins may actually be entirely consumed by that excess of MPs. Therefore, the determination of free annexin levels, rather than total annexin levels, and the computation of a ratio of free annexin levels/circulating PS+MP levels are much more relevant in order to reveal the degree of vascular cell stress in a patient, and to assess the cardiovascular risk. Critically low levels of free functional annexins, or a critically low ratio of free functional annexins/circulating PS+MPs, are therefore linked to widespread cell membrane stress and reveal a higher risk for further severe cardiovascular complications and diseases.

These data therefore open new opportunities for the characterization and stratification of patients at risk for cardiovascular diseases (CVDs), or suffering from cardiovascular diseases, through the use of a new biomarker with optimal decremented potency. The invention may therefore be useful to help predict the occurrence of cardiovascular diseases, and provide patients at risk with better, more appropriate and immediate care.

As the levels of free functional annexins, and more particularly the ratio free functional annexins/circulating PS+MPs truly reflect vascular injury, and more particularly the severity of vascular injury, the inventors propose to use it as a biomarker in stratified medicine, personalized medicine and in healthcare administration. The ability to detect alterations in the levels of free functional annexins, or of the ratio free functional annexins/circulating PS+MPs when individuals are exposed to risk factors in pre-CVD stages, or after a first cardiovascular event, or a first diagnostic of vascular dysfunction or injury, allows to stratify individuals according to the risk that they are exposed to for further vascular compromission and cardiovascular disease. Thus, resources can be focused on those individuals, for which the present biomarker shows that they are at imminent risk for severe complication of vascular injury or vascular dysfunction, or cardiovascular disease.

The present invention therefore relates to an in vitro method for diagnosing a vascular dysfunction or a vascular injury in a subject, said method comprising a step consisting of determining in a plasma sample obtained from said subject the level of free annexins.

The present invention further relates to an in vitro method for determining whether a subject is at risk for severe vasculopathy, cardiovascular complications and cardiovascular diseases, said method comprising a step consisting of determining in a plasma sample obtained from said subject the level of free annexins.

The present invention also encompasses a kit usable in a method according to the invention. This new kit allows the specific assessment of the circulating free functional level of annexins in the plasma sample of a patient. It therefore represents an important improvement, compared to currently available ELISA tests, which do not allow to discriminate between free functional annexin levels and total annexin levels.

The present invention therefore relates to a kit for use in a method according to the invention which comprises:
a first binding member which interacts specifically with an annexin, wherein said first binding partner is preferentially immobilized on a solid support, and
a second binding member which binds annexin and which does not bind annexin previously bound to phosphatidylserine.

Lastly, the invention also relates to a phosphatidylserine antagonist, for use in a method of treatment of severe vasculopathy, cardiovascular complications and cardiovascular diseases in a subject having a decreased level of free annexins as compared to a reference annexin level, wherein the method comprises a determination of the level of free annexins in a plasma sample of said subject.

More particularly, the present invention is defined by the claims.

### BRIEF DESCRIPTION OF THE DRAWINGS

FIG. 1: Scheme illustrating an embodiment of the method for determining the free annexin level according to the invention using RBC MPs produced in vitro. In step one, a plasma sample is added on a solid support (7) wherein first binding members consisting in anti-annexin-A5 antibodies (6) are immobilized. Said first binding members bind both annexin-A5 bound on endogenous PS+MPs (4) as well as free circulating annexin-A5 (5). A first absorbance reading at a wavelength specific for heme (9) is proportional to the amount of heme associated with annexin-A5-covered MPs (1). In step 2, exogenous RBC MPs produced in vitro (2), containing heme (8) and expressing phosphatidylserine (3) at their surface are added on the solid support. A second absorbance reading at a wavelength specific for heme is achieved. This second absorbance value is proportional to the amount of heme associated with endogenous PS+MPs and exogenous PS+MPs. A third absorbance value is obtained by subtracting the first absorbance value to the second absorbance value. This final value is proportional to the fraction of PS+MPs bound to free annexin-A5 initially present in the tested plasma sample.

FIG. 2: A: Free annexin-A5 levels (relative absorbance unit, Abs 398 nm; R.A.U.) as detected in the different groups of the “SCD cohort” (from left to right: controls subject, non SCD sepsis patients, SCD patients at steady state, SCD patients (VOC at early phase), SCD patients (VOC at late phase) SCD patients with sepsis or acute chest syndrome). B-C: Circulated PS+MPs levels (×103 MP/ml) (B) and free annexin-A5 levels (R.A.U.) (C) as estimated in (from left to right) controls subjects, SCD patients at steady state and SCD patients with VOC. D: free annexin-A5/PS+MPs ratio (R.A.U./PS+MPs/μl×1000000) ratio calculated for (from left to right) controls subjects, SCD patients at steady state and SCD patients with VOC. E: Free annexin-A5 levels estimated in the “DIABELYSE” cohort (from left to right) in control non obese patients, control obese patients, arterial hypertensive non obese patients, arterial hypertensive obese patients, diabetic (type 2) non obese patients and diabetic (type 2) obese patients.

FIG. 3: Vaso-occlusive crisis induced by hypoxia. SAD mice were placed in hypoxic conditions overnight and received (see upper trace for 3A-C) or not recombinant annexin-A5 injection. Occurrence of VOCs was monitored through measurement of the mean blood flow velocity (BFV) in the right kidney artery (A) and in pulmonary artery by Echo-Doppler. Cardiac output (B) was further assessed from BFV measurement in pulmonary artery. Heart rate was also measured (C). The results show that annexin injection improves perfusion and cardiac output. (* p<0.05 versus normoxia. $ p<0.05 versus hypoxia)

### DETAILED DESCRIPTION OF THE INVENTION

Definitions

“Diagnosis” and “diagnosing” as used herein generally include a determination of a subject's susceptibility to a disease or disorder, a determination as to whether a subject is presently affected by a disease or disorder, and a prognosis of a subject affected by a disease or disorder.

The terms “treatment”, “treating” or “treat” and the like refer to obtaining a desired pharmacological and/or physiological effect. This effect is preferentially therapeutic in terms of partial or complete stabilization or cure of a disease and/or adverse effects attributable to the disease. Treatment covers any treatment of a disease in a mammal, particularly a human, aimed at inhibiting the disease symptom, (i.e., arresting its development) or relieving the disease symptom (i.e., causing regression of the disease or symptoms). The terms “treatment”, “treating” or “treat” and the like also refer to obtaining a desired pharmacological and/or physiological prophylactic effect in terms or completely or partially preventing a disease or a symptom thereof. It covers therefore any treatment of a disease in a mammal, particularly a human, aimed at preventing the disease or symptom from occurring in a subject which may be at risk, or predisposed to the disease or symptom but has not yet been diagnosed as having it.

The term “plasma sample”, or “sample” encompasses a whole blood, serum, or plasma sample obtained from the patient for the purpose of in vitro evaluation. Preferably the sample according to the invention is a plasma sample. A plasma sample may be obtained using methods well known in the art. Plasma may then be obtained from the plasma sample following standard procedures of the field including, but not limited to, centrifuging the plasma sample, followed by pipetting of the plasma layer. Platelet-free plasma (PFP) can be obtained following appropriate centrifugation. More preferably, the plasma sample obtained from the patient is a platelet free plasma sample.

As used herein, the term “cell microparticles” denotes microparticles (MPs) released into the blood flow by activated or apoptotic cells such as platelets, red blood cells (RBCs), white blood cells, or endothelial cells (Boulanger & Dinat-Georges, 2011 Arterioscler Thromb vas Biol. 2011; 31:2-3; Rubin O et a., Transfus Med Hemother. 2012; 39(5): 342-47). The size of cell MPs ranges from 0.1 μm to 1 μm in diameter, in generally accepted definitions. Typically, said cell MPs express different cell surface markers that are shared with the parent cells. MPs keep a subset of proteins derived from their parental cells, allowing identification of their origin. For example, a red blood cell MP expresses phosphatidylserine at its surface (non cell-specific) and at least one RBC-specific surface marker, such as CD235a.

Sickle cell disease (SCD) is the major genetic disease in France. It was designed as public health priority by UNSECO in 2003 and WHO in 2006. SCD results from a point mutation (HbS) in the globin beta chain and its physiopathology involves an intricate combination of circulating and cardiovascular factors. Indeed, the mutation causes hemoglobin to polymerize, during hypoxic stress mediating drastic and irreversible remodelling of red blood cells (RBCs). In SCD, such as in other hemolytic anemia, sickle RBCs present many unique features including the presence of cell surface phosphatidylserine and adhesion receptors that are normally absent from the surface of mature healthy RBCs. As referred to in the present application SCD refers to the homozygous HbSS phenotype.

Diagnostic and Prognostic Methods According to the Invention:

The present invention relates to a method for diagnosing the occurrence of a vascular dysfunction or a vascular injury in a subject, said method comprising a step consisting of determining in a plasma sample obtained from said subject the level of free annexins.

Vascular dysfunction or vascular injury encompasses endothelial dysfunction or endothelial injury. Endothelial dysfunction according to the invention includes notably loss of endothelial-dependent vasodilatation, apoptosis, leukocyte adhesion, lipid deposition, vasoconstriction, vascular smooth muscle cell (VSMC) proliferation, peripheral resistance, inflammation, thrombosis or ischemic events (Kasprak J D et al., Pharmacol Rep. 2006; 58 Suppl:33-40).

The subject can be any mammalian subject for whom diagnosis, prognosis, treatment, prevention or therapy is desired. Preferably, the subject is a human. The term “subject”, “individual”, and “patient” as mentioned herein are used interchangeably.

The subjects according to the method of the invention may also suffer from at least a pathological condition selected among sickle cell disease, hemolytic anemia, infection, hyperlipidemia, diabetes, glucose intolerance, metabolic syndrome, obesity, hypertension, stress, or a combination of manifestations defining the metabolic syndrome. They may also have or not an unhealthy diet and/or physical inactivity. Said pathological conditions or lifestyle (i.e., diet and physical activity) are known oxidative factors for the endothelium that may lead to endothelium dysfunction or vascular injury.

In SCD patients for example, the sickle erythrocytes display a strong predisposition to aggregate and bind to each other as well as to adhere to the endothelium, to get trapped in small vessels and reduce blood flow. Highly vascularized organs such as kidney, bones and lungs are therefore the seat of disseminated vascular occlusions and recurrent ischemic injuries. VOCs (vaso-occlusions crises) are linked to vaso-occlusions and ischemic events, and participate in recurrent ischemic tissue injury on a chronic basis, resulting in progressive organ damage, multiple organ failure and further cardiovascular damages.

In obese patients, long-term longitudinal studies now indicate that obesity as such not only relates to but independently correlates with coronary atherosclerosis. This relation appears to exist for both men and women with minimal increases in BMI. In a 14-year prospective study, middle-aged women with a BMI >23 but <25 had a 50% increase in risk of nonfatal or fatal coronary heart disease, and men aged 40 to 65 years with a BMI >25 but <29 had a 72% increased risk (Eckel R H. Circulation. 1997; 96: 3248-50). Patients with a BMI above 30 are usually considered as obese.

Endothelial dysfunction and injury as described above therefore encompass damages that precede severe vasculopathy, cardiovascular complications and cardiovascular diseases (CVDs) (Kasprak J D et al., Pharmacol Rep. 2006; 58 Suppl:33-40).

The present invention therefore also relates to a method for determining whether a subject is at risk for severe vasculopathy, cardiovascular complications and cardiovascular diseases, said method comprising a step consisting of determining in a sample (as defined previously, preferably the plasma sample) obtained from said subject the level of free Annexins (i.e., circulating free functional annexins).

In one embodiment the invention relates to a method for determining the risk of vaso-occlusive crises in a subject suffering from sickle cell disease, said method comprising a step consisting of determining, in a plasma sample obtained from said subject, the level of free functional annexins.

In another embodiment, the invention relates to a method for determining the risk for severe vasculopathy, cardiovascular complications and CVDs in a subject suffering from at least one pathological condition selected among hemolytic anemia, infection, hyperlipidemia, diabetes, glucose intolerance, metabolic syndrome, obesity, hypertension, or a combination of manifestations defining the metabolic syndrome said method comprising a step consisting of determining in a sample (as defined above, preferably a plasma sample) obtained from said subject the level of free annexins.

The method of the invention therefore allows stratification of subjects suffering from vascular injury or vascular dysfunction, with respect to their likeliness to develop severe vasculopathy, cardiovascular complications and cardiovascular diseases associated with vascular damage, remodelling or dysfunction (such as vascular dysfunction or vascular injury).

Severe vasculopathy and cardiovascular complications according to the invention include:
Atherogenesis and atherosclerotic plaques, chronic or acute ischemic injury.
Severe complications of sickle cell disease, such as vaso-occlusions, VOCs, severe chest syndrome, ischemic brain injury, retinopathy, priapism.
Degenerative complications and multiple organ failure, such as aseptic osteonecrosis, retinopathy, nephropathy (including renal insufficiency), pulmonary hypertension, pulmonary embolism, cardiac insufficiency, skin ulcerations.
Spell of septicaemia, sudden deafness, severe anemia (blood haemoglobin <6 g/dl).

Typically, CVDs includes stroke, infarction, or peripheral arterial disease.

Annexin proteins are a family of calcium-dependent phospholipid-binding proteins that bind reversibly to membranes through calcium-binding loops in their highly conserved core domains (Gerke V & Moss S E Physiol Rev. 2002; 82(2):331-71). By definition, an annexin protein has to fulfil two major criteria. First, it must be capable of binding in a Ca²⁺-dependent manner to negatively charged phospholipids. Second, it has to contain as a conserved structural element, the so-called annexin repeat, a segment of some 70 amino acid residues.

Each annexin is composed of two principal domains: the divergent NH₂-terminal which confers specificity to annexin intracellular signalling “head” and the conserved COOH-terminal protein core. The latter harbors the Ca²⁺ and membrane binding sites, notably to phosphatidylserine, and is responsible for mediating the canonical membrane binding properties. An annexin core comprises four (in annexin A6 eight) segments of internal and inter-annexin homology that are easily identified in a linear sequence alignment.

As all annexins share the same structural element involved in phospholipid binding, they all bind phosphatidylserine. Therefore all annexins are usable according to the invention. Annexins-A1, -A2, -A4, -A5, -A6 and -A7 are well suited for the method according to the invention and they may be preferred as they have been found in human plasma.

Annexins proteins, which are expressed at the cell surface of red blood cells, such as Annexin-A7 and Annexin-A5, are also particularly preferred.

Annexin-A5 is a member of a protein family whose function is to bind anionic membrane phospholipids and phosphatidylserine (PS) in particular. When membrane is stressed or ruptured, PS is externalized to the surface of membranes (Katrin Fink et al.; Crit Care. 2011; 15(5):R25). PS can then recruit multiple proteins, such as coagulation factors, which PS helps activating. Annexin-A5 functions as a PS inhibitor, passing from cytoplasm to the injured membrane surface, with the help of calcium ions. Annexin-A5 polymerizes in a reticulated network that forms a protective shield on the membrane (Ralf P. Richter et al., Biophysical J. 2005; 89:3372-85; Van Genderen HO et al. Biochim Biophys Acta. 2008; 1783(6):953-63) and isolates it from the extracellular compartment, and blood in particular for vascular and circulating cells. Similar to a healing plaster, the annexin-A5 layer serves to recruit membrane phospholipid that fill in any gap in the membrane, and preserves the cell from lysis and death (McNeil PL1 & Kirchhausen T. Nat Rev Mol Cell Biol. 2005; 6(6):499-505.; Gerke V et al., Nat Rev Mol Cell Biol. 2005; 6(6):449-61.; Bouter et al. Placenta. 2015; 36 Suppl 1:S43-9). Annexin-A5 is thus protective and repairing. Annexin-A5 is strongly expressed in placenta where it fills its protective role in the foetal vascular network (McNeil, 2005; Gerke, 2005; Bouter, 2015). All annexins are able to bind PS, but no other than annexin-A5 has yet been recognized for such protective and reconstructive effects.

Annexin-A5 has a wide panel of potential therapeutic applications in diseases associated to vascular inflammation, with a well described mode of action, and preclinical data. Systemic administration of annexin-A5 has now been analysed in 7 animal modes of cardiovascular injury and disease, in vivo and ex vivo with human cells, including in ischemia/reperfusion episodes and in acute coronary syndromes. Annexin-A5 is able to protect cell integrity and to maintain intact tissue function, such as the survival of a pancreatic implant (Cheng et al. Transplantation. 2010; 90(7):709-16), or the function of a cardiomyocyte (Hale SL et al. Cardiovasc Ther. 2011; 29(4):e42-52.; Gu C et al., Shock. 2015; 44(1):83-9). Annexin-A5 also inhibits atherogenesis (Mark M. et al.; Arterioscler Thromb Vasc Biol. 2011; 31(1):95-101.; Domeij et al., Prostag. Other Lipid Mediat. 2013; 106:72-8.; Wan M et al., Atherosclerosis. 2014; 235(2):592-8) through anti-thrombotic effects (Rand, 2012), and anti-inflammatory effects on T cells (Liu A et al., Arterioscler Thromb Vasc Biol. 2015; 35(1):197-205). The established effects of annexin-A5 include vascular immunomodulation, vulnerable plaque stabilisation and cardiomyocyte protection (Domeij, 2013; Wan, 2014; Liu, 2015).

As used herein, the term “free annexins” relates to a fraction of annexins which is circulating in the blood plasma, and that is functional and fully bio-available to interact with PS+ membranes that it is put in contact with. Thus typically a “free annexin” according to the invention is an annexin, which is not bound to PS+ membranes (and more specifically which is not bound to PS+MPs) and which is not inhibited (for example which is not inhibited by heme).

The level of free annexins determined from a sample (as defined previously, preferably a plasma sample) of a subject, according to the method of the invention as described above, may be further compared to a previous free annexin level obtained from a previous sample from the same subject or preferentially to a reference annexin level detected in a sample of control subjects, or to total annexin levels measured by ELISA technique for instance. Said control subjects may be selected among subjects who underwent or not a vascular injury or a vascular dysfunction. Typically the control subjects are healthy subjects. The reference value can be a threshold or a range. The reference level may be established based upon comparative measurements between patients who underwent a vascular injury or dysfunction and patients who did not undergo a vascular injury or dysfunction.

“Longitudinal” follow-up of patients or “kinetics” of measurements over time can therefore be established according to the method of the invention for each patient. Such longitudinal follow-up allows for the detection of an actively degrading vascular condition in a patient. In this embodiment, the reference level is preferentially a previous free annexin level obtained from a previous sample (as defined previously, preferably a plasma sample) from the same subject.

Typically, a vascular dysfunction or vascular injury is diagnosed in a subject, or the subject is determined to be at risk for severe vasculopathy, cardiovascular complications and cardiovascular disease, when the level of free functional annexins detected in the sample from the subject is decreased as compared to a free annexin reference level.

When the method aims at determining whether a subject is at risk for severe vasculopathy, cardiovascular complications and cardiovascular diseases, the free annexin level determined from the sample (as defined previously, preferably the plasma sample) of the subject can be compared to a reference annexin level obtained in control subjects selected among healthy subjects or among subjects having pathological conditions or lifestyle which are known oxidative factors for the endothelium and that may lead to endothelium dysfunction, as described above.

For example, when the method aims at determining the risk of severe vaso-occlusive crisis complications in a subject suffering from sickle cell disease, the free annexin level determined from the sample of the subject can be compared to a reference annexin level obtained in control subjects selected among healthy subjects or selected among SCD subjects at steady state. Generally SCD subjects are considered at steady state when they are three month away from hospitalization, blood transfusion or hydroxyurea treatment.

The methods for diagnosing a vascular dysfunction or vascular injury in a subject, or for determining whether a subject is at risk for severe vasculopathy, cardiovascular complications and cardiovascular diseases as described above, can further comprise the steps consisting of determining the level of circulating phosphatidylserine positive (PS+) microparticles (PS+MPs) in the sample of the subject and then calculating the ratio free annexins/circulating PS+MPs.

Said calculated ratio free annexins/circulating PS+MPs can be further compared to a reference ratio obtained by dividing a reference annexin level with a reference circulating PS+MPs level. Said reference levels can be obtained as described above from a previous sample (as defined previously, preferably a plasma sample) from the same subject or preferentially from control subjects who underwent or not a vascular injury or dysfunction. Preferentially, said reference levels are obtained in a population of subjects selected among control healthy subjects.

Typically, a vascular dysfunction or a vascular injury is diagnosed in a subject, or the subject is determined to be at risk for severe vasculopathy, cardiovascular complications and cardiovascular diseases, when the ratio free annexin level/circulating PS+MP level is decreased, as compared to the reference ratio free annexin level/circulating PS+MP level.

In one embodiment the methods for diagnosing a vascular dysfunction or vascular injury in a subject, or for determining whether a subject is at risk for severe vasculopathy, cardiovascular complications and cardiovascular diseases as described above, can further, or alternatively to the calculation of the ratio free annexins/circulating PS+MPs, comprise a step consisting of calculating the ratio free annexin level/total annexin level (as measured typically by commercial ELISA). Said free annexin level/total annexin level ratio can also be compared to a reference ratio obtained as mentioned above, from a previous sample from the same subject or preferentially from control subjects who underwent or not a vascular injury or dysfunction.

Typically, a vascular dysfunction or vascular injury may be diagnosed in a subject, or the subject is determined to be at risk for severe vasculopathy, cardiovascular complications and cardiovascular diseases, when the ratio obtained by dividing the free annexin level with the total annexin level is decreased as compared to a reference ratio.

Method for Determining the Levels of Circulating Free Functional Annexin and PS+MPs in a Plasma Sample:

Determination of the level of free annexins in a plasma sample may be performed by any method known in the art.

Preferentially, the level of free functional annexins in a sample (as defined previously, preferably a plasma sample) may be detected through a novel ELISA-like assay coupled to a PS+MP-capture assay, as described below.

In particular, circulating PS+MPs covered with annexins and free circulating annexins may be detected in the sample of the patient using a first binding member capable of selectively interacting with an annexin protein. Said annexin proteins are preferentially annexin proteins which are expressed at the cell surface of red blood cells, such as annexin-A5 and -A7, but any annexin which binds phosphatidylserine is usable according to the invention.

The first binding member may be an anti-annexin antibody, for example an anti-annexin-A5 or an anti-annexin-A7, which may be polyclonal or monoclonal or a fragment or a derivative thereof. In another example, the binding partner may be an aptamer.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g. U.S. Pat. No. 4,946,778) can be adapted to produce single chain antibodies. Antibodies useful in practicing the present invention also include fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity. For example, phage display of antibodies may be used. In such a method, single-chain Fv (scFv) or Fab fragments are expressed on the surface of a suitable bacteriophage, e.g., M13. Briefly, spleen cells of a suitable host, e. g., mouse, that has been immunized with a protein are removed. The coding regions of the VL and VH chains are obtained from those cells that are producing the desired antibody against the protein. These coding regions are then fused to a terminus of a phage sequence. Once the phage is inserted into a suitable carrier (e.g., bacteria), the phage displays the antibody fragment. Phage display of antibodies may also be provided by combinatorial methods known to those skilled in the art. Antibody fragments displayed by a phage may then be used as part of an immunoassay.

Aptamers are a class of molecules that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by Exponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consist of conformationally constrained antibody variable regions displayed by a platform protein, such as E. coli Thioredoxin A, that are selected from combinatorial libraries by two hybrid methods (Colas et al, 1996).

The aforementioned assay preferentially involves immobilization of the first binding member (i.e., antibody or aptamer) on a solid support thus forming an ELISA test support. Solid supports which can be used in the practice of the invention include substrates such as nitrocellulose (e. g., in membrane or microtiter well form); polyvinylchloride (e. g., sheets or microtiter well plates); polystyrene latex (e.g., beads or microtiter plates); polyvinylidine fluoride; diazotized paper; nylon membranes; activated beads, magnetically responsive beads, and the like. Well plates, notably having an opaque black wall usable for fluorometry are well suited according to the invention. More preferentially, an ELISA based assay is used, wherein the wells of a microtiter plate are coated with a set of anti-annexin antibodies.

Immobilization of the first binding member on the support may be achieved by any techniques known in the state of the art, allowing typically for covalent linkage of the binding member on the support (see for example Angles-Cano et al., Journal of Immunological Methods 1984; 69:115-27). Typically, glutaraldehyde based protocols are used, wherein glutaraldehyde (preferentially 0.5-5%) is polymerized on the solid support for optimal attachment of the binding member. The protein-free sites, available for protein crosslinking can also be neutralized using for example an ethanolamine buffer solution.

A sample (as defined previously, preferably a plasma sample) is then added to the ELISA-test support as described above. The sample is preferentially a platelet-free plasma sample in order to avoid possible saturation of annexins with activated PS+ platelets. After a period of incubation sufficient for allowing interaction between MPs and the first binding member, the plasma can be eliminated, the ELISA-test support is washed and a detectable secondary binding member is added. Said secondary binding member is selected in order to specifically bind annexins that are not bound to phosphatidylserine. Thus this secondary binding member must be able to detect specifically the free annexins bound on the first binding member while no binding the annexins bound on PS+MPs.

Preferably, said detectable secondary binding member comprises exogenous phospholipidic vesicles expressing phosphatidylserine (PS+ vesicles) at their surface. Such PS+ vesicles are therefore capable of specifically binding free annexin proteins bound on the first binding member (i.e., annexin proteins which are not engaged with PS+MPs).

PS+ vesicles according to the invention can be artificial double layer vesicles expressing phosphatidylserine in their external phospholipidic layer. Such vesicles can be assembled according to well-known techniques of the art (Camus S et al., Blood 2015 125(25):3805-14).

PS+ artificial vesicles may be detectably labelled with a detectable molecule or substance, such as a fluorescence molecule (for example: fluorescein isothiocyanate, (FITC), phycoerythrin (PE), or indocyanine (Cy5)), a radioactive molecule or any other labels known in the art.

The measured signal obtained from the detectable PS+ vesicles reflects the binding of PS+ vesicles to free functional annexins bound on the first binding member and can therefore be directly correlated to the amount of free annexins in the sample of the subject.

More preferentially, the amount of free annexins in the sample is reflected by the difference between said measured signal obtained from the second binding member and the background signal measured in the same conditions, before addition of the second binding member on the ELISA-test support.

The assay is also well suited to evaluate the level of circulating MPs though detection of cell-free heme and hemoglobin associated to annexin-covered MPs in the sample (as defined previously, preferably a plasma sample). This may be assessed by taking a first absorbance reading, before addition to the ELISA-test support of the second binding member, at wavelengths specific for heme (i.e., preferably around 398-415, 540 or 575 nm). Said absorbance measurement is therefore proportional to the amount of endogenous heme associated to annexin-covered MPs in the plasma sample.

In these conditions, the used second binding member preferentially comprises heme containing exogenous PS+ vesicles. For example, artificial PS+ vesicles formed according to classical techniques used for multilamellar vesicles (MLV) or large unilamellar (LUV) can be charged with heme or any hemoprotein (Camus S et al., Blood 2015 125(25):3805-14) and used, wherein heme or any hemoprotein constitutes the detectable molecule as mentioned previously.

The binding of exogenous PS+ vesicles to “free annexins” can be assessed by taking a second absorbance reading at wavelengths specific for heme (i.e., preferably around 398-415, 540 or 575 nm).

This second absorbance reading therefore combines the absorption provided by endogenous heme associated to annexin-covered MPs from the plasma sample (corresponding to the first absorbance reading) and the additional absorbance related to the heme contained in the exogenous PS+ vesicles. Therefore the difference between this second absorbance reading and the first absorbance reading (thus considered as background) represents the extra binding of the exogenous PS+ vesicles onto the free annexins immune-adsorbed on the first binding member. This value is thus proportional to the amount of functional MPs-free annexins in the sample (as defined previously, preferably a plasma sample).

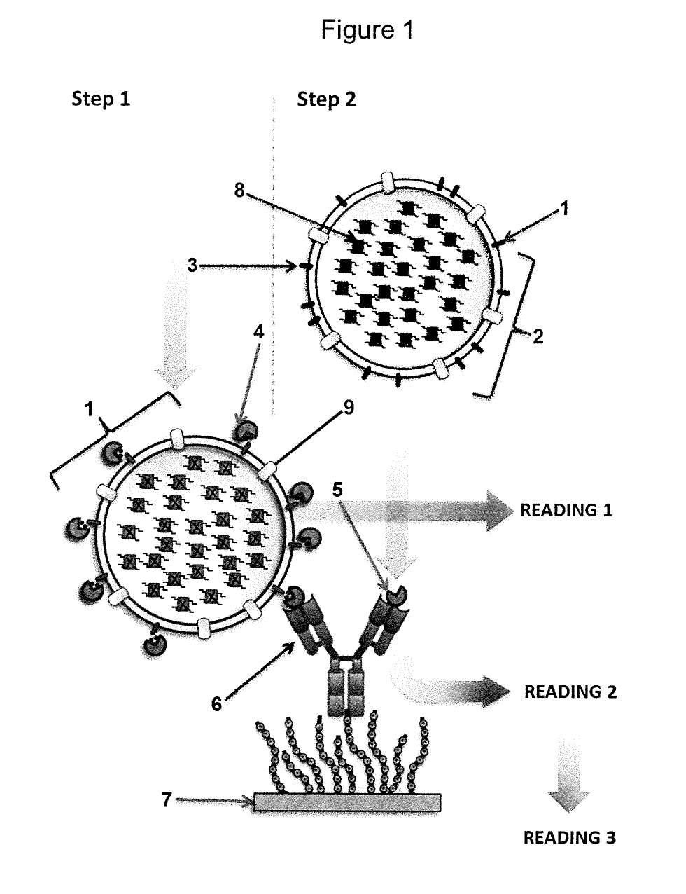

In one embodiment of the method for determining the level of circulating MPs-free annexin and the level of circulating PS+MPs in a sample (as defined previously, preferably a plasma sample), according to the invention, the exogenous MP+ vesicles can also be microparticles (MPs) produced in vitro and purified from red blood cells (RBCs) (see also FIG. 1) according to known techniques of the art such as described in WO2012/120131 or in Camus S M et al., Blood 2012; 120(25):5050-58. Briefly, a stock of purified MPs can be produced from RBCs sorted by gradient centrifugation. The RBCs preparation is preferably depleted in peripheral blood mononuclear cells and neutrophils using appropriate separation media. RBC preparation is then activated and subjected to low speed centrifugation. Supernatant is then collected and MPs may be further concentrated using ultracentrifugation.

RBCs MPs can be isolated according to standard methods of the art (see notably Camus S M et al., Blood 2012; 120(25):5050-58). For example a classical method consists in collecting the population of MPs which is present in the supernatant of the cells and using a specific binding member directed against a specific molecules expressed at their surface such as an annexin protein (more preferentially annexin-A5 or annexin-A7) or the surface marker CD235a, wherein MPs are bound by said binding member to said molecule.

Typically the specific binding member can be an antibody or a fragment thereof or an aptamer, as described above. Said binding member may be labelled with a detectable molecule as also described previously.

Methods of flow cytometry are preferred methods for collecting MPs, for example FACS (Fluorescence-activated cell sorting) or magnetic beads. Size exclusion columns or filters may also be used to purify MPs of specific sizes out of the cell supernatant.

A population of exogenous MPs as mentioned above (whether artificial vesicles or MPs produced from RBCs) can easily be conserved in an appropriate medium and stored as a bank of artificial vesicles or cell MPs. Typically, cell MPs can be stored frozen at low temperature such as −20° C., or at −80° C. for long term storage, and artificial vesicles can be stored frozen or at up to +4° C., before loading with heme.

Determination of the Level of Circulating Phosphatidylserine Positive (PS+) Microparticles (PS+MPs) in a Sample:

It is noted that when required in a method according to the invention, determination of the level of circulating phosphatidylserine positive (PS+) microparticles (PS+MPs) in a sample (as defined previously, preferably a plasma sample), in order to calculate the free annexin/circulating PS+MPs ratio, is preferentially achieved as described above. Preferentially, the amount of circulating PS+MPs in the sample of the subject is estimated by FACS after labelling of the MPs using an antibody or a fragment thereof directed against an annexin (for example annexin-A5 or annexin-A7) or against a surface marker, such as CD235a.

The use of an antibody (or a fragment thereof) directed against an annexin may lead to underestimation of the total amount of circulating MPs in the sample, as MPs wherein the phosphatidylserine is fully covered by annexins may not be detected. Therefore the use of an antibody directed against a surface marker, for example CD235a may be preferred.

Kit Usable in a Method According to the Invention:

Another object of the invention relates to a kit usable in a method as previously described and comprising means for detecting free functional annexin in a sample (as defined previously, preferably a plasma sample).

Typically said kit comprises:
a) a first binding member which interacts specifically with an annexin protein, and
b) a second binding member which binds annexins and which does not bind annexins bound to phosphatidylserine, as previously described.

A kit according to the invention may also comprise, as a separate component, a specific component designed to help immobilize the first binding partner (typically an antibody) on a solid support. This component is preferably a solution of glutaraldehyde (0.5-5% preferred). A solution used for blocking the protein-free sites such as a solution of ethanolamine may also be added. Alternatively, said first binding member is immobilized on a solid support as previously described.

Preferentially, a second binding member is a PS+ vesicle as described above.

A kit according to the invention may also comprise, as a separate component, an additional binding member that interacts specifically with an annexin protein (notably annexin-A5 or annexin-A7) or a surface marker, notably a surface marker of red blood cells MPs such as CD235a. Typically said additional binding member is an antibody.

Method of treatment according to the invention:

The present invention also relates to a method of treating vascular dysfunction or vascular injury as well as severe vasculopathy, cardiovascular complications and cardiovascular diseases in a subject which has been diagnosed with vascular injury or which has been determined at risk for severe vasculopathy, cardiovascular complications and cardiovascular diseases comprising:
the detection of the free annexin level in a sample (as defined previously, preferably a plasma sample) of said subject,
the comparison of said detected free annexin level with a reference level as described previously, and
the administration of a phosphatidylserine antagonist or an inhibitor of phosphatidylserine receptor expression to said subject, when the detected level of free annexin in the plasma sample of the subject is decreased as compared to the reference free annexin level.

Said method may further comprise:
the detection of the circulating PS+MP level;
the calculation of the ratio free annexin level/circulating PS+MPs;
the comparison of said free ratio annexin level/circulating PS+MPs with a reference ratio as previously defined; and
the administration of a phosphatidylserine antagonist (for example an annexin such as a recombinant annexin) or an inhibitor of phosphatidylserine receptor expression, when said free annexin level/circulating PS+MPs calculated ratio is decreased as compared to the reference ratio.

As used herein the term “phosphatidylserine receptor antagonist” refers to any agent that inhibits the binding of phosphatidylserine to phosphatidylserine receptor. Said antagonist may be selected form the group consisting of small molecule, antibodies, aptamers, and polypeptides.

The phosphatidylserine receptor antagonist may consist in an antibody or antibody fragment directed against phosphatidylserine or phosphatidylserine receptor. As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

In another embodiment the phosphatidylserine receptor antagonist is an aptamer as described previously.

In another embodiment, the phosphatidylserine receptor antagonist is a polypeptide, especially a polypeptide having the ability to bind posphatidylserine. Typically said polypeptide binds the polar head of phosphatidylserine in a calcium dependant way, such as an annexin or any one of the polypeptides mentioned below.

Said polypeptide may be recombinant or not and may consists in, or derive from, a polypeptide selected from the group consisting of annexins, (notably annexin-A1, -A2, -A3, -A4, -A5, -A6, -A7, -A8, -A9 or -A10, notably annexin-A5), annexin peptides, developmental endothelial locus-1 (Del-1) protein, synaptotagmin I, lactadherin, T cell immunoglobulin mucin 1 and 4 (TIM-1, TIM-4), c-carboxyglutamic acid (Gla) containing proteins such as vitamin K-dependent blood coagulation factors (which include notably Factor II, Factor VII, Factor IX and Factor X, the anticoagulant proteins C and S, and the factor X-targeting protein Z) .

In one embodiment, the phosphatidylserine receptor antagonist is an annexin-A5 or a modified annexin-A5 polypeptide.

The modified annexin-A5 polypeptide can be a polymer of annexin-A5 that has an increased effective size. It is believed that the increase in effective size results in prolonged half-life in the vascular compartment. One such modified annexin-A5 can be a dimer of annexin-A5. In one embodiment, the dimer of annexin V is a homodimer of annexin-A5. Said homodimer of human annexin-A5 may be prepared in using well-established methods of recombinant DNA technology. The annexin-A5 molecules of the homodimer are joined through peptide bonds to a flexible linker. In some embodiments, the flexible linker contains a sequence of amino acids flanked by a glycine and a serine residue at either end to serve as swivels. The linker preferably comprises one or more such “swivels”. Preferably, the linker comprises 2 swivels which may be separated by at least 2 amino acids, more particularly by at least 4 amino acids, more particularly by at least 6 amino acids, more particularly by at least 8 amino acids, more particularly by at least 10 amino acids. Preferably, the overall length of the linker is 5-30 amino acids, 5-20 amino acids, 5-10 amino acids, 10-15 amino acids, or 10-20 amino acids. The dimer can fold in such a way that the convex surfaces of the monomer which bind phosphatidylserine, can both gain access to externalized phosphatidylserine. Flexible linkers are well known in the art. Typically a homodimer of annexin-A5 is diannexin as described in Kuypers F A, Larkin S K, Emeis J J, Allison A C. Interaction of an annexin-A5 homodimer (Diannexin) with phosphatidylserine on cell surfaces and consequent antithrombotic activity. Thromb Haemost. 2007 March; 97(3):478-86.

In another embodiment of the invention, modified annexin-A5 polypeptide may consist on a recombinant annexin V expressed with, or chemically coupled to, another protein such as the Fc portion of immunoglobulin. Such expression or coupling increases the effective size of the molecule, preventing the loss of annexin-A5 from the vascular compartment and prolonging the half-life of said modified annexin-A5 polypeptide.

In a particular embodiment the polypeptide is a functional equivalent of phosphatidylserine receptor. As used herein, a “functional equivalent of phosphatidylserine receptor” is a compound which is capable of binding to phosphatidylserine, thereby preventing its interaction with phosphatidylserine receptor. The term “functional equivalent” includes fragments, mutants, and muteins of phosphatidylserine receptor. The term “functionally equivalent” thus includes any equivalent of phosphatidylserine receptor obtained by altering the amino acid sequence, for example by one or more amino acid deletions, substitutions or additions such that the protein analogue retains the ability to bind to phosphatidylserine. Amino acid substitutions may be made, for example, by point mutation of the DNA encoding the amino acid sequence.

Functional equivalents include molecules that bind phosphatidylserine and comprise all or a portion of the extracellular domains of phosphatidylserine receptor. Typically, said functional equivalents may comprise binding domain of phosphatidylserine receptor or a portion thereof.

The functional equivalents include soluble forms of the phosphatidylserine receptor. A suitable soluble form of these proteins, or functional equivalents thereof, might comprise, for example, a truncated form of the protein from which the transmembrane domain has been removed by chemical, proteolytic or recombinant methods.

Preferably, the functional equivalent is at least 80% homologous to the corresponding protein. In a preferred embodiment, the functional equivalent is at least 90% homologous as assessed by any conventional analysis algorithm such as for example, the Pileup sequence analysis software (Program Manual for the Wisconsin Package, 1996).

The term “a functionally equivalent fragment” as used herein also may mean any fragment or assembly of fragments of phosphatidylserine receptor that binds to phosphatidylserine.

Accordingly the present invention provides a polypeptide capable of inhibiting binding of phosphatidylserine receptor to phosphatidylserine, which polypeptide comprises consecutive amino acids having a sequence which corresponds to the sequence of at least a portion of an extracellular domain of phosphatidylserine receptor, which portion binds to phosphatidylserine.

Functionally equivalent fragments may belong to the same protein family as the human phosphatidylserine receptor identified herein.

By “protein family” is meant a group of proteins that share a common function and exhibit common sequence homology. Homologous proteins may be derived from non-human species. Preferably, the homology between functionally equivalent protein sequences is at least 25% across the whole of amino acid sequence of the complete protein. More preferably, the homology is at least 50%, even more preferably 75% across the whole of amino acid sequence of the protein or protein fragment. More preferably, homology is greater than 80% across the whole of the sequence. More preferably, homology is greater than 90% across the whole of the sequence. More preferably, homology is greater than 95% across the whole of the sequence.

The polypeptides of the invention may be produced by any suitable means, as will be apparent to those of skill in the art. In order to produce sufficient amounts of polypeptides of the invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeasts and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In specific embodiments, it is contemplated that polypeptides used in the therapeutic methods of the present invention may be modified in order to improve their therapeutic efficacy. Such modification may be used to decrease toxicity, increase circulatory time, or modify biodistribution. For example, the toxicity of potentially important therapeutic compounds can be decreased significantly by combination with a variety of drug carrier vehicles that modify biodistribution.

Preferentially, a phosphatidylserine antagonist is an annexin, notably a recombinant annexin (typically any one of the annexins A1 to A10, notably recombinant annexin-A5 or -A7), the developmental endothelial locus-1 (Del-1) protein, which may be recombinant or not, or any one of the Gla containing proteins, recombinant or not (notably Factor II, Factor VII, Factor IX and Factor X, the anticoagulant proteins C and S, and the factor X-targeting protein Z).

The present invention also relates to a phosphatidylserine antagonist as described above, for use in a method of treatment of severe vasculopathy, cardiovascular complications and cardiovascular disease in a subject having a decreased level of free annexin as compared to a reference annexin level, wherein the method comprises a determination of the level of free annexin in a plasma sample of the subject.

Said method may further comprises the steps consisting of determining the circulating PS+MP level in said plasma sample and calculating the ratio free annexin/circulating PS+MPs.

### EXAMPLES

Materials and Methods

Circulating, functional and bioavailable annexin-A5 was measured using an in house-designed immunosorbant assay.

Antibody Immobilization

An anti-human annexin-A5 antibody (Affymetrix eBioscience, #BMS147; 10 ug/ml) was immobilized on the bottom of a 96 well plate (preferably with opaque black walls for fluorometry; Dominique Dutscher #655090), according to previously published protocols (Angles-Cano E, 1984, Journal of Immunological Methods 69:115-127). Briefly, glutaraldehyde was left to polymerize onto the clear polystyrene bottom by leaving 100 μl of 2.5% glutaraldehyde for 2 h at 22° C. After rinsing with water (H₂O), the anti-human annexin-A5 antibody (10 μg/ml in 0.1 M BicNa, pH 8.5 buffer) was loaded into the wells and left to incubate at 4° C. for 12 to 18 hours. The antibody was then attached to the immobilized glutaraldehyde polymers. The wells were then saturated with ethanolamine buffer (0.3 M, pH 7.4) for 2 h at 22° C. in order to neutralize any potential site left available for protein cross-linking. After emptying and rinsing 3 times with in HEPES buffer (10 mM pH 7.4, 0.15 M NaCl), the plates were blocked with bovine serum albumin (2 mg/ml) in HEPES buffer (10 mM, 0.05% Tween-20, pH 7.4), and ready to perform the assay or stored at 4° C.

Plasma Microparticle Immunoadsorption

The plates were emptied, and platelet-free plasma (5%; i.e.; 5 μl in 95 μl of PBS-0.9 mM Ca²⁺) was placed in the wells and incubated for 1 h at 37° C. Platelet-free plasma (depleted by 2× centrifugations at 2500 g, for 10 min at 22° C.) was preferred in order to prevent possible saturation of annexins by activated PS+ platelets.

Plasma Microparticle Immunoadsorption Measurement

The plasma-supernatant was then eliminated and the plates were rinsed twice with phosphate buffer saline (PBS-0.9 mM Ca²⁺; Ca²⁺ was present at all steps in order to insure maintain and facilitate PS-annexin interactions). The assay is particularly well suited to evaluate the levels of circulating cell-free heme and hemoglobin associated to annexin-covered microparticles in plasma. This can be assessed by taking absorbance readings at wavelengths specific for heme (i.e.;. preferably 398 nm or 540 nm or 575 nm). A first measurement was taken at this step, when absorbance is proportional to the amount of heme associated to endogenous annexin-A5-covered bodies in the circulation. (*) indicate p<0.05 vs matched Controls, (#) p<0.05 vs SCD (steady state), ($) p<0.05 vs SCD (VOC early) (see FIG. 1).

Exogenous Red Blood Cell Microparticle Adsorption

A stock of purified microparticles was produced and purified from red blood cells sorted by gradient centrifugation using classical methods. The red blood cell preparation was preferably depleted in peripheral blood mononuclear cells and neutrophils using appropriate separation media (here, Granulosep, Eurobio #CMSMOP01). Red blood cells adjusted to 40% hematocrit in PBS-0.9 mM Ca2+, were stimulated with calcimycin (A23287, 1 μM, Sigma-Aldrich #C9275). After low speed centrifugation, supernatant were collected and titrated in microparticles by FAGS after labeling with fluorescent rh-annexin-A5 (Roche Diagnostics #11828681001) versus calibrated microbeads (FlowCount™, Beckman-Coulter, #7547053), and size was confirmed by Nanoparticle Tracking Analysis (NTA; Malvern NanoSight™). Red blood cell supernatants rich in MP were diluted to 5% (or down to about 13680 MP/μl; and/or Abs398 nm between 0.05 and 0.2 RAU) in PBS-5 mM Ca2+ and incubated in the wells for 1 h at 37° C. (The calcium concentration was increased to maximize interactions between adsorbed membrane-free annexin-A5 and exogenous microparticle phosphatidylserines.)

Exogenous Red Blood Cell Microparticle Adsorption Measurement

The plates were rinsed twice with phosphate buffer saline (PBS-0.9 mM Ca²⁺) to eliminate unbound microparticles. An absorbance reading (reading 2) was taken at a wavelength specific for heme (i.e., preferably 398-415 nm or 540 nm or 575 nm). This reading 2 combines the absorption provided by endogenous heme associated to annexin-covered bodies from the circulation, and the additional absorbance provided by the binding of the exogenous red blood cell microparticles, which contain heme naturally. Here, the endogenous levels (reading 1) were considered as background and subtracted (from reading 2). The difference in absorbance represents the extra binding of the purified microparticles onto the immunoadsorbed annexin-A5, and it is thus proportional to the amount of functional and MP-free annexin-A5 in plasma (see FIG. 1). All data were expressed in relative absorbance units (R.A.U.). (*) indicate p<0.05 vs matched Controls. (#) p<0.05 vs SCD (VOC early).

Optimization of quantification and the selection of the plasma and MP dilutions were performed using serial dilutions of rh-annexin-A5 (Becton Dickinson, #556416) adsorbed directly onto the plastic bottom of the wells (0.1 to 10 ng/ml), serial dilutions of plasma, and serial dilutions of red blood cell MP.

Calculation of the Ratio Free Annexin-A5/PS+MPs

A ratio of the free annexin-A5 concentration divided by the PS+MP concentration was calculated. The free annexin-A5 concentration was expressed in relative absorbance units (Abs980 nm in the example) and divided by the PS+MP concentration expressed in MP/μl. The resulting ratio was multiplied by 1,000,000 for ease of presentation. The resulting unit is a purely relative index and does not represent any specific plasma concentration.

Patient Cohorts

Human Subjects and the HEMIR Cohort:

SCD patients, sepsis (non SCD) patients, and healthy (non SCD) control volunteers of 18 years of age or more, were enrolled after informed consent and approval by the ethical committees of the French Scientific Research Ministry, as collection protocol DC-2011-1450-HEMIR. All subjects were without recent blood transfusion or known infection. Patients with hemoglobin SS (HbSS) had previously been identified by high-pressure liquid chromatography by a hematologist on the faculty of Administration Publique-Hopitaux de Paris (AP-HP, Paris, France). To avoid confounders related to SCD genotype, we focused on patients with HbSS (under 15% patients with HbSβ0 thalassemia).

HEMIR Included:

(1) Patients with HbSS (SCD patient) in steady state (usual state of health, over 3 months away from VOC, hospitalization or emergency department visit) presenting to the sickle cell program outpatient clinics of Tenon and George Pompidou hospitals in Paris, or Avicenne hospital in Bobigny (AP-HP). Patients underwent routine blood testing as indicated by their clinical care. Demographics and other available laboratory measurements were obtained aspart of the ongoing care of the patients. Clinical data such as treatment with hydroxy-urea (HU) were obtained at inclusion. HU-treated patients were excluded.

(2) SCD patients during the “early” phase of vaso-occlusive crises, within 3 days of hospitalization,

(3) SCD patients during the “late” phase of vaso-occlusive crises, within 5 and 10 days of hospitalization,

(4) SCD patients during the “early” phase of vaso-occlusive crises, within 3 days of hospitalization and presenting sepsis or an acute chest syndrome (ACS) combined with pulmonary infection.

(5) Non-SCD volunteers hospitalized in acute care for sepsis (n=6),

(6) Healthy African descent control subjects matched in sex-, age- and ethnics distribution (phototypes 5 and 6) with HbSS patients, without known hemolytic anemia, were recruited by the medical center of the Caisse Primaire d'Assurance Maladie de la Seine-St Denis in Bobigny.

Human Subjects and the DIABELYSE Cohort :

The DIABELYSE cohort included:

Diabetic (type 2, DT2), hypertensive and obese patients as well as a group of healthy control volunteers of 18 years of age or more, who were enrolled after informed consent and approval by the ethical committees of the French Scientific Research Ministry, as collection protocol DC-2011-1480-DIABELYSE. All subjects were free of known coagulopathy or recent blood transfusion or infection.
Patients were considered diabetic when their glycemia was above >1.26 g/l (with double check), or when they have been treated for diabetes.
Obese patients were defined by a body mass index (BMI) above 30.
Patients with arterial hypertension (HTA) were defined by a arterial tension above 140/40, or by their hypotensive treatment.

Vaso-Occlusive Crises (VO) in SAD Mice:

VOC were induced in SAD mice under hypoxic conditions. SAD mice were placed in hypoxic conditions (9% O2) Overnight (16-18 hours).

We characterized the induction of VOC in SAD transgenic mice (18, 19) according to our previously published method (Bonnin P et al., Ultrasound Med Biol 2008; 34(7):1076-1084; Sabaa N et al., J Clin Invest 2008; 118(5):1924-1933; Camus et al., Blood 2012 120:5050-5058), in response to the hypoxic conditions. Briefly, mice were anesthetized with isoflurane and monitored to prevent any cardiorespiratory depression. Mice were shaved and placed in the decubitus position on a heating blanket (38° C.). We used a Vivid 7 echograph (GE Medical Systems®, Horten, Norway) equipped with a 12-MHz linear transducer (12L). The ultrasound probe was placed on the left side of the abdomen for examination of renal arteries, or in left lateral decubitus for cardiac output acquisition with transducer on the chest. Data were transferred on-line to an EchoPAC ultrasound image analysis workstation (GE Medical Systems®). Two-Dimensional ultrasound imaging of the abdomen in left-sided longitudinal B-mode allowed kidney width and height measurements. Color-coded blood flow detection by Doppler enabled renal arteries to be localized. A pulsed Doppler spectrum was recorded and peak systolic, end-diastolic and time-average mean blood flow velocity (BFV) were measured in the renal artery, with Doppler beam angle correction. To calculate cardiac output, spatial flow profiles were analyzed in parasternal long-axis B-mode images of the pulmonary artery and BFV were measured as above. The following formula was applied: CO=[(V^{mean}0.60). (Tr. (Dpa/2)²)], where CO is the cardiac output in ml/minute, V^{mean} is the mean time-averaged BFV in cm/s and Dpa is the pulmonary artery diameter in cm. Kidney sizes and BFV were determined by the same investigator, as means of 5 to 8 measurements. Repeatability for cardiac output measurements was verified. We controlled and confirmed that kidney size was similar between SAD and wild type mice. After sacrifice, kidneys were dissected, dehydrated, mounted in paraffin, sectioned and stained by Masson trichrome. Vascular congestion was observed by phase-contrast microscopy, as large erythrocyte aggregates occluding kidney capillaries and larger vessels.

Results

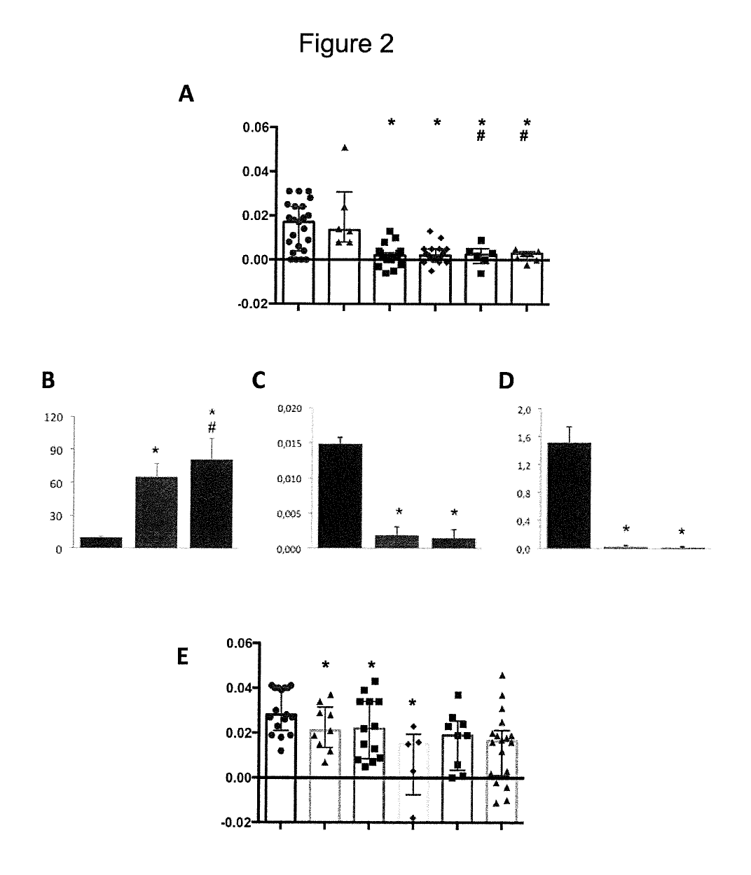

The results, obtained in the HEMIR cohorts, show that some endogenous annexin-A5 remains bioavailable for PS binding in the circulation of healthy volunteers (0,015 R.A.U.; See FIG. 2A, ANOVA p<0.0001) such that a sudden rise in circulating PS+MP levels, or PS externalization in blood and vascular cells, might thus be targeted by this excess free annexin-A5.

MP-free functional annexin-A5 levels were strongly decreased (0.002 R.A.U.; p<0.05 vs. SCD (steady state)), often below detectable thresholds, in all patients with SCD (0.002 R.A.U. at steady state; 0.001 R.A.U. during CVO (early); 0.001 R.A.U. during CVO (late); 0.002 R.A.U. in SCD with infection; p<0.05 vs. non-SCD controls). The drop was independent from sepsis and pulmonary infection, in both SCD and control samples (0.020 R.A.U. in controls with sepsis).

The levels of PS+MPs, free annexin-A5 as well as the ratio free annexin-A5/PS+MPs were then assessed and compared among control subjects (group 6), SCD patients at steady state (group 1) and SCD patients who underwent VOCs (groups 2, 3 and 4).

The levels of circulating PS+MPs (FIG. 2B) were significantly increased in SCD patients at steady state (65.0 MP/ul) vs. control groups (9.83 MP/ul; p<0.05 vs. controls). Circulating PS+MP levels rose again significantly in SCD (VOC) patients (80.4 MP/ul; p<0.05 vs. SCD steady state).

On the contrary, the free annexin-A5 levels were drastically reduced in SCD patients (steady) (FIG. 2C; 0.002 versus 0.015 R.A.U. in controls; p<0.05), and further decreased in SCD (VOC) patients (0.001 R.A.U.; p<0.05 vs. SCD steady state).

Varying degree of rise in circulating PS+MPs as well as a dramatic and concomitant drop in MP-free functional annexin-A5 levels therefore occurred in all SCD patients. Therefore, critically low levels of MP-free functional annexin-A5 and a critically low ratio of MP-free functional annexin-A5/circulating PS+MP marked all SCD patients (1.51; 0.029; 0.018 arbitrary units, respectively), particularly during VOC (p<0.05 vs. SCD steady state) (see FIG. 2D). These results therefore illustrate that a critically low ratio of MP-free annexin-A5 /circulating PS+MPs may thus serve to identify, characterize and stratify patients at high risk of CVD, whether in SCD cohorts.

The results also indicate that SCD patients, at steady state and more so during acute phase VOC, may be particularly fragile despite the beneficial management of pain and the approaching end of hospitalization. SCD patients might remain with low natural defenses against pro-inflammatory, pro-aggregant and pro-thrombotic circulating PS+MPs, and at high cardiovascular risks in case of a new rise.

Similar results were obtained in the DIABELYSE cohorts as defined previously. Indeed, free annexin-A5 could readily be detected in non-obese, non-HTA, non-DT2 control volunteers (0.030 R.A.U. See FIG. 2E, ANOVA p=0.0042). As shown for SCD patients, the free annexin levels were drastically decreased in patients presenting at least one pathological condition selected among obesity (0.022 R.A.U.), arterial hypertension (0.016 R.A.U. and 0.008 R.A.U. when obese; p<0.05 vs. non-obese controls) or diabetes (0.023 R.A.U. and 0.018 R.A.U. when obese; p<0.05 vs. non-obese controls). It is noted that the free annexin level is further decrease when patients are hypertensive and obese or diabetic and obese (p<0.05 vs. non-obese).

It has further been shown that Annexin-A5 supplementation cures vaso-occlusions in SAD mice.

A bolus of recombinant human annexin-A5 was injected intraveinously in SAD mice, wherein hypoxic VOCs have been induced, and kidney reperfusion and cardiac output were monitored.

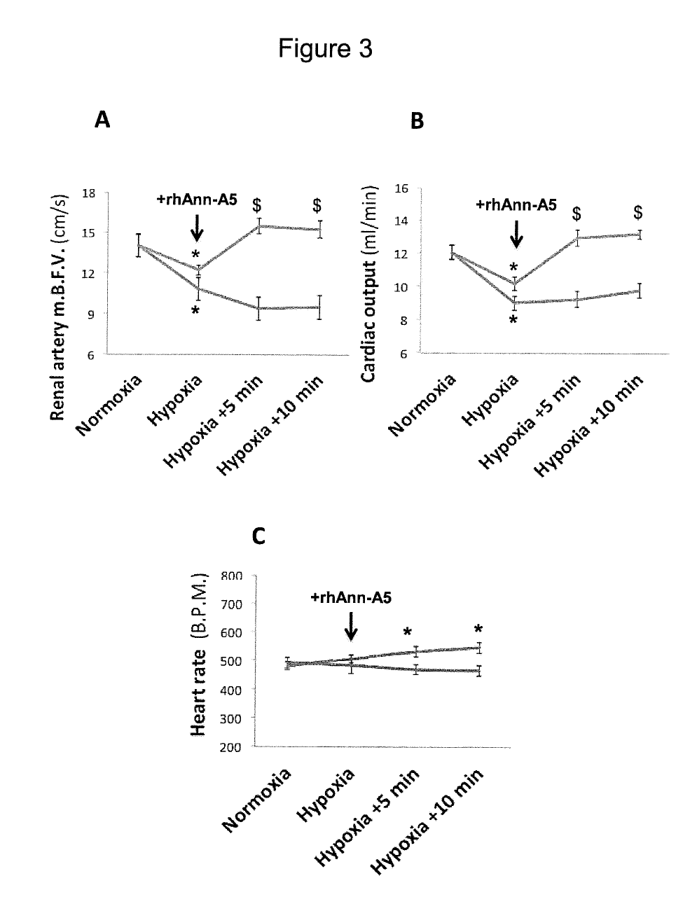

The results showed (see FIG. 3A-C) an excellent improvement of kidney perfusion; i.e.: a rapid augmentation of renal artery blood flow velocity was observed. Similarly the mean blood flow velocity in pulmonary artery also rapidly and significantly increased, illustrating the improvement of cardiac output. Little or no significant effect on heart rate (control) was observed.

### CONCLUSIONS

The inventors have therefore shown that the free annexin-A5 level is drastically decreased in patients with vascular dysfunction and at high cardiovascular risks such as SCD patients or obese patients (further presenting or not diabete or arterial hypertension).

Their results indicate that the level of free circulating annexin may be used as a useful biomarker for vascular dysfunction diagnosis as well as for determining the patients which are at high risks for cardiovascular diseases. Critically low levels of free functional annexins, or a critically low ratio of free functional annexins/circulating PS+MPs, are therefore linked to widespread cell membrane stress and reveal a higher risk for further severe cardiovascular complications and diseases.

The determination of free annexin levels, rather than total annexin levels, and the computation of a ratio of free annexin levels/circulating PS+MP levels are therefore much more relevant in order to reveal the degree of vascular cell stress in a patient, and to assess the cardiovascular risks.

These data therefore open new opportunities for the characterization and stratification of patients at risk for cardiovascular diseases (CVDs), or suffering from cardiovascular diseases.

Lastly, the results also demonstrate that administration of a phosphatidylserine antagonist, such as an annexin (here annexin A5), cures vaso-occlusive crisis.

1. A method for diagnosing the occurrence of a vascular dysfunction or a vascular injury in a subject, said method comprising a step consisting of determining in a plasma sample obtained from said subject the level of free annexin.

2. A method for determining whether a subject is at risk for severe vasculopathy, cardiovascular complications and cardiovascular disease, said method comprising a step consisting of determining in a plasma sample obtained from said subject the level of free annexin.

3. The method according to claim 1 wherein the annexin is annexin-A5.

4. The method according to claim 1 3, wherein the subject is suffering from at least a pathological condition selected among sickle cell disease, hemolytic anemia, infection, hyperlipidemia, diabetes, glucose intolerance, metabolic syndrome, obesity, hypertension or stress and/or have an unhealthy diet or physical inactivity.

5. The method according to claim 1 further comprising the steps consisting of determining the level of circulating phosphatidylserine positive (PS+) microparticles (MPs) in the plasma sample obtained from said subject and calculating the ratio free annexin/circulating PS+MPs.

6. (canceled)

7. The method according to claim 1 wherein said method comprises using a kit comprising:
a first binding member which interacts specifically with an annexin protein, wherein said first binding partner is immobilized on a solid support, and
a second binding member which binds annexin and which does not bind annexin bound to phosphatidylserine.

8. The method according to claim 7 wherein the second binding member is a PS+ vesicle.

9. The method according to anyone of claim 7 wherein said kit further comprises as a separate component, an additional binding member that interacts specifically with annexin or with a surface marker of red blood cells MPs.

10. The method according to claim 9 wherein the additional binding member is an anti-annexin antibody or an anti-CD235a antibody.

11. A method of treatment of severe vasculopathy, cardiovascular complications and cardiovascular disease in a subject having a decreased level of free annexin as compared to a free annexin reference level, wherein the method comprises a step of determining the free annexin level of in a plasma sample of said subject and a step of administering a phosphatidylserine antagonist to said patient.

12. The method according to claim 11, for the treatment of severe vasculopathy, cardiovascular complications and cardiovascular disease in a subject having a decreased ratio of free annexin/circulating PS+MPs as compared to a free annexin/circulating PS+MPs reference ratio, wherein the method further comprises the steps consisting of determining the circulating PS+MPs level in said plasma sample and calculating the free annexin/circulating PS+MPs ratio.

13. The method according to claim 11, wherein said phosphatidylserine antagonist is a polypeptide which binds the polar head of phosphatidylserine in a calcium dependant way.

14. The method according to claim 13, wherein said polypeptide is selected from the group consisting of annexins, annexin peptides, developmental endothelial locus-1 (Del-1), synaptotagmin 1, lactadherin, T cell immunoglobulin mucin 1 and 4 (TIM-1, TIM-4), and c-carboxyglutamic acid (Gla) containing proteins.

15. The method according to claim 2, wherein the annexin is annexin-A5.

16. The method according to claim 2, wherein the subject is suffering from at least a pathological condition selected among sickle cell disease, hemolytic anemia, infection, hyperlipidemia, diabetes, glucose intolerance, metabolic syndrome, obesity, hypertension or stress and/or have an unhealthy diet or physical inactivity.

17. The method according to claim 2 further comprising the steps consisting of determining the level of circulating phosphatidylserine positive (PS+) microparticles (MPs) in the plasma sample obtained from said subject and calculating the ratio free annexin/circulating PS+MPs.

18. The method according to claim 2, wherein said method comprises using a kit comprising:
a first binding member which interacts specifically with an annexin protein, wherein said first binding partner is immobilized on a solid support, and
a second binding member which binds annexin and which does not bind annexin bound to phosphatidylserine.

19. The method according to claim 18 wherein the second binding member is a PS+ vesicle.

20. The method according to claim 18, wherein said kit further comprises as a separate component, an additional binding member that interacts specifically with annexin or with a surface marker of red blood cells MPs.

21. The method according to claim 20, wherein the additional binding member is an anti-annexin antibody or an anti-CD235a antibody.
